(19) United States
(12) Patent Application Publication
(10) Pub. No.: US 2013/0165782 A1
(43) Pub. Date: Jun. 27, 2013

(54) ULTRASONIC DIAGNOSIS APPARATUS AND CONTROL PROGRAM OF THE SAME

(71) Applicant: GE MEDICAL SYSTEMS GLOBAL TECHNOLOGY COMPANY, LLC, Waukesha, WI (US)

(72) Inventor: Tsutomu Yawata, Tokyo (JP)

(73) Assignee: GE MEDICAL SYSTEMS GLOBAL TECHNOLOGY COMPANY, LLC, Waukesha, WI (US)

(21) Appl. No.: 13/720,678

(22) Filed: Dec. 19, 2012

(30) Foreign Application Priority Data
Dec. 26, 2011 (JP) 2011-283377

(51) Int. Cl. A61B 8/08; A61B 8/00; A61B 8/14

(52) U.S. Cl. CPC A61B 8/5223; A61B 8/14; A61B 8/463; A61B 8/54
USPC 600/440

(57) ABSTRACT

An ultrasonic diagnosis apparatus is provided. The ultrasonic diagnosis apparatus includes a measuring unit configured to perform a measurement on an instructed part in at least one measurement ultrasonic image, a storing unit configured to store the at least one measurement ultrasonic image on which the measurement is performed, a statistical processing unit configured to perform a statistical process on results of measurements performed a plurality of times on the same object in the same subject by the measuring unit, and an image display control unit configured to read a display ultrasonic image stored in the storing unit and display the display ultrasonic image together with a statistical value obtained by the statistical processing unit.

### CROSS REFERENCE TO RELATED APPLICATIONS

This application claims the benefit of Japanese Patent Application No. 2011-283377 filed Dec. 26, 2011, which is hereby incorporated by reference in its entirety.

### BACKGROUND OF THE INVENTION

The present invention relates to an ultrasonic diagnosis apparatus capable of performing a measurement in an ultrasonic image and a control program of the same.

An ultrasonic diagnosis apparatus capable of measuring the size of a tumor mass, the size of a fetus, and the like is disclosed in, for example, Japanese Unexamined Patent Application Publication No. 2008-284263. At the time of performing a measurement, for example, the operator makes a cursor displayed on an ultrasonic image, places the cursor on an object to be measured, and performs a measurement. In some cases, an obtained measurement value is used for a report.

To assure accuracy of a measurement value, the measurement is often performed a plurality of times. In this case, an average value may be calculated from measurement values obtained by the plurality of times of measurements. In the case of performing a statistical process for obtaining an average value or the like from results of the plurality of times of measurements, a statistical value such as the average value may be displayed on a display unit of the ultrasonic diagnosis apparatus. However, when only a numerical value is displayed, in some cases, it is difficult to easily grasp the object of the measurement from the displayed average value. Therefore, it is preferable for the operator that not only a statistical value but also an ultrasonic image in which the measurement is performed are displayed.

### BRIEF DESCRIPTION OF THE INVENTION

An ultrasonic diagnosis apparatus is provided. The ultrasonic diagnosis apparatus includes a measuring unit which performs a measurement on an indicated part in an ultrasonic image, a storing unit which stores the ultrasonic image on which the measurement is performed, a statistical processing unit which performs a statistical process on results of measurements of a plurality of times performed on the same object in the same subject by the measuring unit and an image display control unit which reads an ultrasonic image stored in the storing unit and displays it together with a statistical value obtained by the statistical processing unit.

According to the above-described apparatus, since a statistical value obtained by the statistical processing unit is displayed together with the ultrasonic image on which the measurement is performed, it is convenient for the operator.

### BRIEF DESCRIPTION OF THE DRAWINGS

FIG. 1 is a block diagram illustrating an example of an embodiment of an ultrasonic diagnosis apparatus.

FIG. 2 is a block diagram illustrating a display control unit in the ultrasonic diagnosis apparatus depicted in FIG. 1.

FIG. 3 is a block diagram illustrating a control unit in the ultrasonic diagnosis apparatus depicted in FIG. 1.

FIG. 4 is a diagram illustrating a display unit in which an ultrasonic image is displayed.

FIG. 5 is a diagram illustrating a display unit in which a cursor for performing a measurement is displayed in an ultrasonic image.

FIG. 6 is a diagram illustrating a display unit in which an ultrasonic image stored in a memory and an average value of measurement values are displayed.

### DETAILED DESCRIPTION OF THE INVENTION

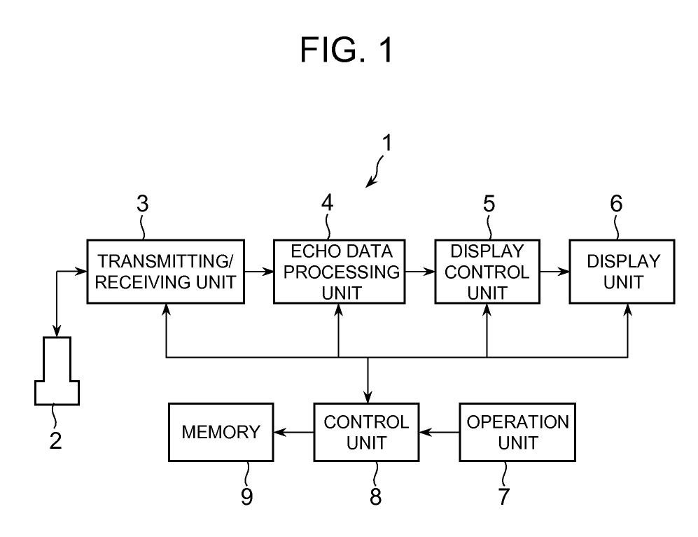

Hereinafter, an exemplary embodiment of an ultrasonic diagnosis apparatus will be specifically described with reference to the drawings. As illustrated in FIG. 1, an ultrasonic diagnosis apparatus 1 has an ultrasonic probe 2, a transmitting/receiving unit 3, an echo data processing unit 4, a display control unit 5, a display unit 6, an operation unit 7, a control unit 8, and a memory 9.

The ultrasonic probe 2 has a plurality of ultrasonic transducers (not illustrated) disposed in an array, transmits an ultrasonic wave to a subject by the ultrasonic transducers, and receives an echo signal of the ultrasonic wave.

The transmitting/receiving unit 3 supplies an electric signal, which is for transmitting an ultrasonic wave from the ultrasonic probe 2 under predetermined scan conditions, to the ultrasonic probe 2 on the basis of a control signal from the control unit 8. The transmitting/receiving unit 3 performs signal processes such as A/D conversion, phasing/adding process, and the like on an echo signal received by the ultrasonic probe 2, and outputs the echo data subjected to the signal processes to the echo data processing unit 4.

The echo data processing unit 4 performs processes for generating an ultrasonic image on the echo data outputted from the transmitting/receiving unit 3. For example, the echo data processing unit 4 performs a B-mode process including a logarithmic compression process and an envelope detecting process to generate B-mode data. The echo data processing unit 4 may perform another process for generating an ultrasonic image other than the B-mode process.

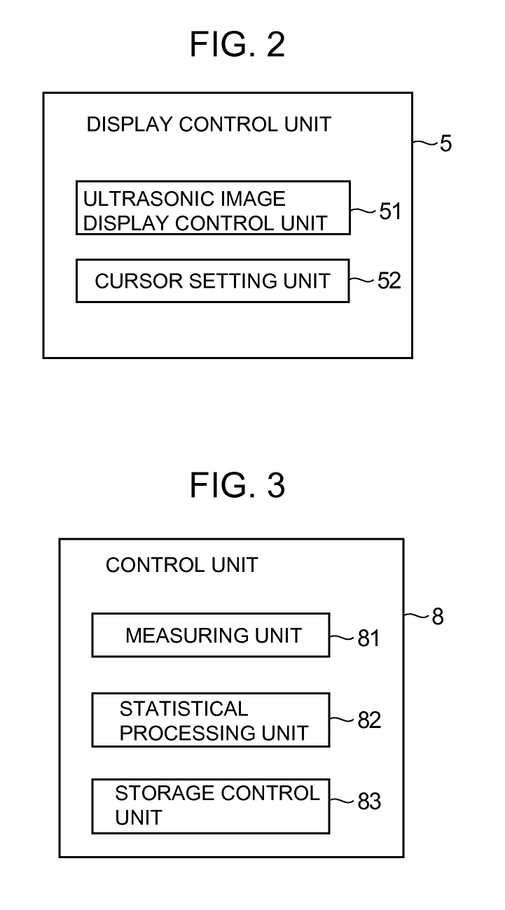

As illustrated in FIG. 2, the display control unit 5 has an ultrasonic image display control unit 51 and a cursor setting unit 52. The ultrasonic image display control unit 51 performs a scan conversion to the data supplied from the echo data processing unit 4 by a scan converter to generate ultrasonic image data. The ultrasonic image display control unit 51 displays an ultrasonic image on the display unit 6 on the basis of the ultrasonic image data.

For example, the display control unit 5 performs scan conversion to the B-mode data to generate B-mode image data and displays a B-mode image on the basis of the B-mode image data.

Data such as B-mode data before a scan conversion to ultrasonic image data by the scan converter is also referred to herein as raw data.

As will be described later, the ultrasonic image display control unit 51 reads an ultrasonic image stored in the memory 9 and displays the image together with an average value of measurement values obtained by a measuring unit 81 (refer to FIG. 3) on the display unit 6 (image display control function). The ultrasonic image display control unit 51 is an example of an embodiment of an image display control unit.

The cursor setting unit 52 sets a measurement cursor C (refer to FIG. 5) on the display unit 6 on the basis of an input in the operation unit 7. For example, the cursor setting unit 52 sets the measurement cursor C on an ultrasonic image UG displayed on the display unit 6 on the basis of an operation of a pointing device (which will be described later) by an operator. The measurement cursor C is indicated by an X mark in the example. The measurement cursor C is constructed by two cursors C1 and C2. The measurement cursor C is an example of an embodiment of an indicator.

The display unit 6 is an LCD (Liquid Crystal Display), a CRT (Cathode Ray Tube), or the like. The operation unit 7 includes a keyboard and a pointing device (not illustrated) used by the operator to enter an instruction and information.

The control unit 8 is a CPU (Central Processing Unit) and has, as illustrated in FIG. 3, the measuring unit 81, a statistical processing unit 82, and a storage control unit 83. The measuring unit 81 performs a measurement on a part indicated by the cursor C in the ultrasonic image UG displayed on the display unit 6. In the example, the measuring unit 81 measures the distance between the measurement cursors C1 and C2. The measuring unit 81 is an example of an embodiment of a measuring unit.

For example, by the measuring unit 81, the size of a tumor mass, the size of a fetus, and the like are measured. The measurement is performed a plurality of times on the same object in the same subject. In the case where an object to be measured is a moving object such as the heart, a measurement under the same condition (the same time phase in the case of the heart) is counted as one measurement, and the measurement is performed a plurality of times.

The statistical processing unit 82 performs statistical process on results of the measurements of the plurality of times performed on the same object in the same subject by the measuring unit 81. For example, the statistical processing unit 82 calculates an average value of results of measurements of a plurality of times. The statistical processing unit 82 is an example of the embodiment of a statistical processing unit.

A statistical process is performed on a plurality of measurement values. A statistical process includes processes such as calculation of an average value of the plurality of measurement values, specification of a maximum value and a minimum value in the plurality of measurement values, or calculation of a variance and a standard deviation of the plurality of measurement values. The statistical processing unit 82 may specify the maximum value and the minimum value and calculate a variance and a standard deviation.

The storage control unit 83 stores an ultrasonic image into the memory 9. The details will be described later. The storage control unit 83 is an example of an embodiment of a storage control unit.

The control unit 8 reads a control program stored in the memory 9 to execute other functions in units in the ultrasonic diagnosis apparatus 1.

The memory 9 is, for example, a semiconductor memory such as an RAM (Random Access Memory). In the memory 9, for example, a result of a measurement performed by the measuring unit 81, an ultrasonic image on which a measurement is performed, and the like are stored. The memory 9 is an example of an embodiment of a storing unit.

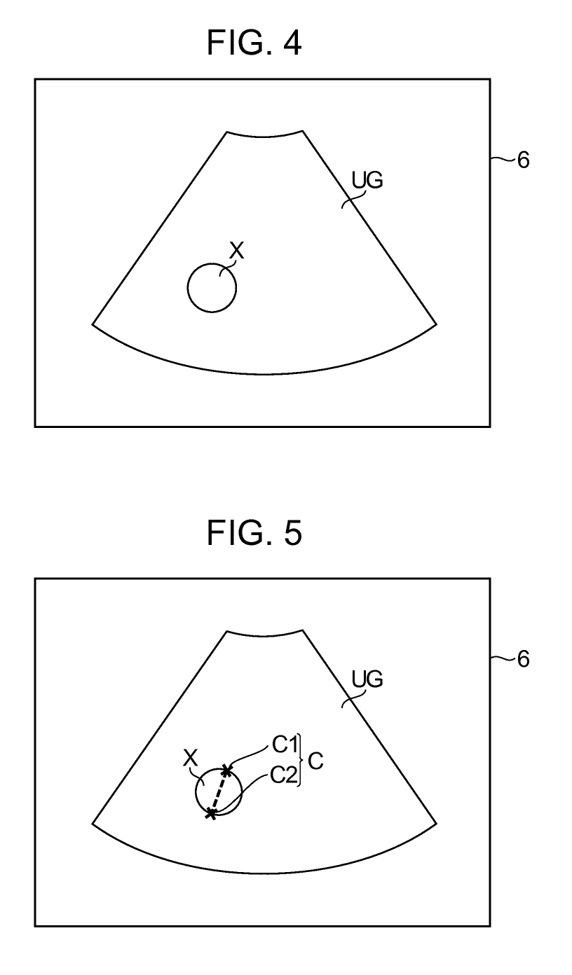

The operation of the ultrasonic diagnosis apparatus 1 of the embodiment will be described. First, an ultrasonic wave is transmitted/received by the ultrasonic probe 2 to make the ultrasonic image UG displayed on the display unit 6 as illustrated in FIG. 4.

In the ultrasonic image UG, the tumor mass X is seen. In the example, the diameter of the tumor mass X is measured. More specifically, when the operator enters an input to display the cursor C in the operation unit 7, the cursor setting unit 52 displays the cursor C on the display unit 6, as illustrated in FIG. 5. When the cursors C1 and C2 are placed on the contour of the tumor mass X and the operator presses a measurement button in the operation unit 7, the measuring unit 81 measures the distance between the cursors C1 and C2. The measurement of the distance is performed on the ultrasonic image UG and the ultrasonic image UG is frozen. The operator sets the cursors C1 and C2 on the tumor mass X and performs measurement a plurality of times. The measurement values are stored in the memory 9.

The storage control unit 83 stores at least one of the frozen ultrasonic images UG used in the measurements of a plurality of times into the memory 9. The ultrasonic image UG stored in the memory 9 is an ultrasonic image in which the cursor C is displayed.

The storage control unit 83 may store the ultrasonic image UG into the memory 9 on the basis of an input of the operation unit 7 entered by the operator. The storage control unit 83 may automatically store the ultrasonic image UG into the memory 9 at a preset predetermined measurement timing (for example, a first measurement and a last measurement). The ultrasonic image UG may be stored in the form of ultrasonic image data or in the form of raw data.

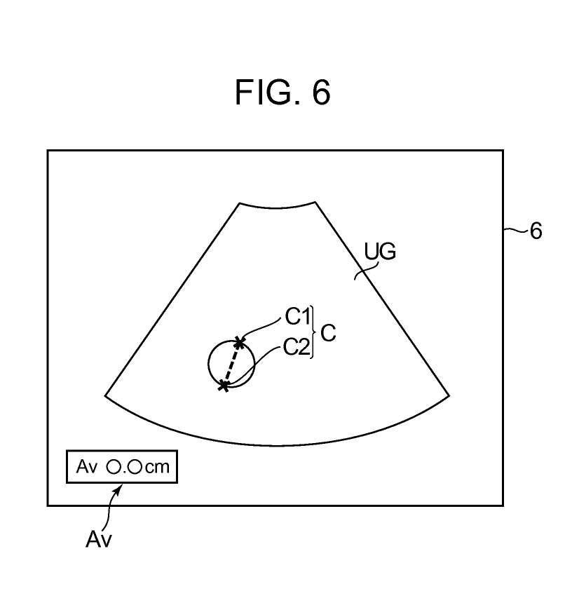

After completion of the measurements of the plurality of times, the statistical processing unit 82 calculates the average value Av of measurement values in a series of measurements stored in the memory 9. The ultrasonic image display control unit 51 reads the ultrasonic image UG stored in the memory 9 and displays it together with the average value Av on the display unit 6, as illustrated in FIG. 6. In the case where there is an input in the operation unit 7 by the operator, the ultrasonic image display control unit 51 may reads the ultrasonic image UG from the memory 9 and display it together with the average value Av.

The ultrasonic image display control unit 51 may display the maximum value and the minimum value or the variance and standard deviation together with the ultrasonic image UG. Those values may be displayed together with the average value Av.

In the ultrasonic diagnosis apparatus 1 of the embodiment, the average value Av is displayed together with the ultrasonic image UG stored in the memory 9. Therefore, it is convenient for the operator.

Although exemplary embodiments are described herein, the invention can be modified without departing from the scope and spirit of the disclosure. For example, the object to be measured in the foregoing embodiment is an example object, and the object is not limited to the above-described object.

In the case where a variance and a standard deviation are large, there is the possibility that the measurement values are various and accurate measurements were not performed. Consequently, in the case where a variance or a standard deviation exceeds a predetermined threshold, the fact may be notified by displaying the message or generating a warning sound. The ultrasonic diagnosis apparatus 1 may have a notifying unit performing the notification.

1. An ultrasonic diagnosis apparatus comprising:
a measuring unit configured to perform a measurement on an instructed part in at least one measurement ultrasonic image;
a storing unit configured to store the at least one measurement ultrasonic image on which the measurement is performed;
a statistical processing unit configured to perform a statistical process on results of measurements performed a plurality of times on the same object in the same subject by the measuring unit; and
an image display control unit configured to read a display ultrasonic image stored in the storing unit and display the display ultrasonic image together with a statistical value obtained by the statistical processing unit.

2. The ultrasonic diagnosis apparatus according to claim 1, wherein the display ultrasonic image is at least one of the measurement ultrasonic images on which the measurement is performed.

3. The ultrasonic diagnosis apparatus according to claim 1, further comprising a storage control unit configured to store ultrasonic images in the storing unit based on an input of an operating unit by an operator.

4. The ultrasonic diagnosis apparatus according to claim 2, further comprising a storage control unit configured to store ultrasonic images in the storing unit based on an input of an operating unit by an operator.

5. The ultrasonic diagnosis apparatus according to claim 1, further comprising a storage control unit configured to store ultrasonic images in the storing unit at a predetermined timing which is preset.

6. The ultrasonic diagnosis apparatus according to claim 2, further comprising a storage control unit configured to store ultrasonic images in the storing unit at a predetermined timing which is preset.

7. The ultrasonic diagnosis apparatus according to claim 5, wherein the predetermined timing is a measurement timing of any of the performed measurements.

8. The ultrasonic diagnosis apparatus according to claim 6, wherein the predetermined timing is a measurement timing of any of the performed measurements.

9. The ultrasonic diagnosis apparatus according to claim 1, wherein the measuring unit is configured to perform a measurement on a part indicated by an indicator displayed on the ultrasonic image.

10. The ultrasonic diagnosis apparatus according to claim 1, wherein the statistical value is any of an average value, a maximum value, a minimum value, a variance, and a standard deviation.

11. The ultrasonic diagnosis apparatus according to claim 2, wherein the statistical value is any of an average value, a maximum value, a minimum value, a variance, and a standard deviation.

12. The ultrasonic diagnosis apparatus according to claim 3, wherein the statistical value is any of an average value, a maximum value, a minimum value, a variance, and a standard deviation.

13. The ultrasonic diagnosis apparatus according to claim 4, wherein the statistical value is any of an average value, a maximum value, a minimum value, a variance, and a standard deviation.

14. The ultrasonic diagnosis apparatus according to claim 5, wherein the statistical value is any of an average value, a maximum value, a minimum value, a variance, and a standard deviation.

15. The ultrasonic diagnosis apparatus according to claim 6, wherein the statistical value is any of an average value, a maximum value, a minimum value, a variance, and a standard deviation.

16. The ultrasonic diagnosis apparatus according to claim 7, wherein the statistical value is any of an average value, a maximum value, a minimum value, a variance, and a standard deviation.

17. The ultrasonic diagnosis apparatus according to claim 8, wherein the statistical value is any of an average value, a maximum value, a minimum value, a variance, and a standard deviation.

18. The ultrasonic diagnosis apparatus according to claim 9, wherein the statistical value is any of an average value, a maximum value, a minimum value, a variance, and a standard deviation.

19. A control program of an ultrasonic diagnosis apparatus, the control program configured to make a computer execute:
a measuring function of performing a measurement on an indicated part in a measurement ultrasonic image;
a storage controlling function of storing the measurement ultrasonic image on which the measurement is performed into a storing unit;
a statistical processing function which performs a statistical process on results of measurements performed a plurality of times on the same object in the same subject by the measuring function; and
an image display control function of reading a display ultrasonic image stored in the storing unit and displaying the display ultrasonic image together with a statistical value obtained by the statistical processing function.

20. A method for use with an ultrasonic diagnosis apparatus, said method comprising:
performing a measurement on an indicated part in a measurement ultrasonic image;
storing the measurement ultrasonic image on which the measurement is performed in a storing unit;
performing a statistical process on results of measurements performed a plurality of times on the same object in the same subject;
reading a display ultrasonic image stored in the storing unit; and
displaying the display ultrasonic image together with a statistical value obtained by performing the statistical process.
